(19) United States
(12) Patent Application Publication
(10) Pub. No.: US 2025/0120746 A1
(43) Pub. Date: Apr. 17, 2025

(54) METHOD FOR MANUFACTURING EARLY TREATMENT APPLIANCE FOR GUIDING MANDIBULAR GROWTH AND RECONSTRUCTION WITH MAXILLARY OCCLUSION STRUCTURE

(71) Applicant: Guangzhou Yaqi Dental Medical Technology Co., Ltd, Guangzhou (CN)

(72) Inventors: Ying CHEN, Guangzhou (CN); Xiaoyan GUO, Guangzhou (CN)

(73) Assignee: Guangzhou Yaqi Dental Medical Technology Co., Ltd, Guangzhou (CN)

(21) Appl. No.: 18/750,920

(22) Filed: Jun. 21, 2024

(30) Foreign Application Priority Data
Oct. 13, 2023 (CN) 202311333209.5

(51) Int. Cl. A61B 17/66; A61B 34/10

(52) U.S. Cl. CPC A61B 17/663; A61B 34/10; A61B 2034/105; A61B 2034/107

(57) ABSTRACT

A method for manufacturing an early treatment appliance for guiding mandibular growth and reconstruction with a maxillary occlusal structure is provided. A maxillary arch is arranged by determining a functional occlusal plane, a treatment target position of a maxillary first molar, a treatment target position of a maxillary central incisor, a treatment target positions of canine to bicuspid in a middle section of the maxillary arch, and a maxillary arch form, and a target mandibular arch is determined based on a centric relation jaw position and the target maxillary arch, and then the mandibular occlusion reconstruction is guided by adjusting the mandibular arch or arranging a jaw pad based on the condition of the child. The maxillary arch structure can be restored to a normal range conforming to the age and growth pattern structure of an individual while the pathogenesis is controlled.

### CROSS-REFERENCE TO RELATED APPLICATIONS

This application claims priority to Chinese Patent Application No. CN 202311333209.5, filed on Oct. 13, 2023. The content of the aforementioned applications, including any intervening amendments thereto, is incorporated herein by reference.

### FIELD

The present application relates to the technical field of early orthodontic treatment for children, and in particular to a method for manufacturing an early treatment appliance for guiding mandibular growth and reconstruction with a maxillary occlusal structure.

### BACKGROUND

Permanent dentition refers to the arrangement of normal permanent teeth that have grown or are growing in human mouth. An occlusal relationship of normal permanent dentition, maxillofacial muscles and temporomandibular joint are in a stable and coordinated relationship with each other. Children with mixed dentition are in a period of rapid growth and development, their occlusal relationship will change significantly as deciduous teeth being replaced by the permanent teeth. The maxillofacial muscles will also change to adapt to the changes of the occlusal relationship, leading to a new imbalance. Therefore, it is necessary to promote development of facial bones, especially mandibles, and reconstruction and reshaping of the temporomandibular joint, so that the three can reach a new stage of balance.

However, during the six or seven years of mixed dentition, children's maxillary arch structure may not be well-developed and growth and development of the occlusal relationship may deviate from a normal track due to various reasons, leading to abnormal development of muscles, bones and temporomandibular joints, and resulting in increasingly serious maxillofacial deformities and oral malocclusion.

At present, most orthodontists diagnose and treat the oral malocclusion by taking the mandible as a dominant part. This method is feasible for patients with the permanent dentition. However, for children around 9 years old who are in a period of rapid growth and development, the growth of their maxillary bones is basically completed, while the mandibular bones are in the growth and development peak at the age of 10 to 13 years old, during which period the position, shape and structure of the mandible undergo obvious changes. Therefore, during the early orthodontic treatment of mixed dentition and permanent dentition for children before the mandible is fully grown, if diagnosis, treatment and product design are based on the mandible, there will be disadvantages such as unclear diagnostic and treatment ideas, cumbersome step-by-step treatment, low product design efficiency and poor treatment effect.

On the other hand, since the full-cycle early orthodontic treatment for children currently available on the market spans a wide age range, clinical work is difficult to implement, the treatment cycle is long, the cost is high, and it takes much time before the effect starts, resulting in low compliance of children, high expenses for parents, and physical and mental exhaustion of doctors, making it difficult to promote the early orthodontic treatment effectively in a long term. Moreover, currently in the market, clinical doctors are guided by manufacturers to carry out the early orthodontic treatment mainly with a certain type of orthodontic appliance, there is a lack of professional connection with the manufacturer's product design in a personalized diagnosis of clinical cases and a comprehensive selection of the orthodontic appliance. The product design fails to truly take into consideration the growth and development of the occlusal function of children with mixed dentition, and cannot achieve accurate, efficient and safe orthodontic treatment by correcting the abnormal occlusal function of the children. Since there are few doctors who can apply the relevant knowledge of functional gnathology to the early orthodontic treatment, the knowledge of functional gnathology has not been fully applied in the interaction between early orthodontic clinicians and manufacturers to form standardized treatment modes and product design and manufacture.

### SUMMARY

To this end, a method for manufacturing an early treatment appliance for guiding mandibular growth and reconstruction with a maxillary occlusal structure is provided according to the present application, to solve the problem in the conventional technology that the early treatment appliance with the mandible as a dominate part is not suitable for child patients with mixed dentition and early-stage permanent dentition.

To achieve the above objectives, the present application provides the following technical solutions.

In a first aspect, a method for manufacturing an early treatment appliance for guiding mandibular growth and reconstruction with a maxillary occlusal structure includes:
step 1: determining a target functional occlusal plane based on a skull base structure, an axis-orbital plane and a maxillary palatal plane, where the target functional occlusal plane is within a normal range of angle value between an SN reference plane and an FH reference plane, and divides a vertical distance between a maxilla and a mandible to make it symmetrically pass through mandibular foramina or central points of mandibular bodies on both sides;
step 2: determining a treatment target position of a maxillary first molar based on the skull base structure and a maxillary bone structure;
step 3: determining a treatment target position of a maxillary central incisor;
step 4: determining treatment target positions of canine to bicuspid in a middle section of a maxillary arch;
step 5: determining a dental arch form based on a craniofacial bone form, a facial form and a central incisor crown shape, where the dental arch form is pointed round, oval or square round;
step 6: arranging the target maxillary arch based on the target functional occlusal plane, the treatment target position of the maxillary first molar, the treatment target position of the maxillary central incisor, the treatment target positions of the canine to bicuspid in the middle section of the maxillary arch, and the dental arch form;
step 7: determining a target mandibular arch based on a centric relation jaw position and the target maxillary arch;
step 8: adjusting a mandibular position according to a child's condition, and using a functional appliance combined with invisible braces or a one-piece jaw pad to guide the mandible to match the maxilla at an occlusal reconstruction position to form the target mandibular arch.

Preferably, the step 1 includes:
step 101: determining ideal positions of a maxillary point A and a mandibular point B based on the skull base structure, the axis-orbital plane and the maxillary palatal plane, to obtain a target anterior cranial base (AB) plane;
step 102: setting an inclination of the functional occlusal plane to make it be perpendicular to the target AB plane, and performing adjustment within the normal range of the angle value between the SN reference plane and the FH reference plane based on the skull base structure and growth pattern;
step 103: dividing the vertical distance between the maxilla and the mandible to make it symmetrically pass through the central points of the mandibular bodies or the mandibular foramina on both sides, to obtain the final target functional occlusal plane.

Preferably, the step 2 includes:
step 201: determining a width of a buccal wall of a mesiobuccal cusp of the maxillary first molar based on a width of the skull base structure and a width of the maxillary base;
step 202: determining a sagittal position, a vertical position and a mesiodistal inclination of the maxillary first molar based on that a tooth long axis of the maxillary first molar passing through the mesiobuccal cusp is perpendicular to the target functional occlusal plane and an extension line of the tooth long axis of the maxillary first molar passing through the mesiobuccal cusp passes through a KR point; where when determining the sagittal position, the vertical position and the mesiodistal inclination of the maxillary first molar, distances from condylar centers on both sides to the mesiobuccal cusps of the maxillary first molars are set to be the same;
step 203: determining a buccolingual torque of the maxillary first molar based on an ideal curve of Wilson;
step 204: determining a rotation degree of the maxillary first molar based on an arch width, an arch curvature and a position of a contralateral canine;
step 205: obtaining the treatment target position of the maxillary first molar based on the width, the sagittal position, the vertical position, the mesiodistal inclination, the buccolingual torque and the rotation degree.

Preferably, the step 3 includes:
step 301: determining a radius range of a midpoint of a palatine fossa of the maxillary central incisor based on an equilateral triangle formed by a distance between the condylar centers on both sides of a cone beam computed tomography (CBCT) image;
step 302: determining a sagittal position and a vertical position of the maxillary central incisor of an ideal treatment target position of the maxillary arch based on the radius range, the target functional occlusal plane, a compensating curve and a maxillary central incisor closing axis;
step 303: obtaining child's current condyle path data based on a CBCT glenoid fossa image;
step 304: determining child's current incisal guidance and condylar guidance based on the child's age and the condyle path data;
step 305: determining a treatment target position incisal path of the maxillary central incisor based on the incisal guidance, the condylar guidance, and the growth pattern;
step 306: determining an inclination of the maxillary central incisor based on the midpoint of the palatine fossa of the maxillary central incisor, the treatment target position incisal path of the maxillary central incisor, and palatine anatomical form of the maxillary central incisor;
step 307: obtaining the treatment target position of the maxillary central incisor by fine-tuning the sagittal position and the vertical position of the maxillary central incisor and the inclination of the maxillary central incisor with reference to a correlation between a height of contour of the maxillary central incisor and a facial profile, angles between a long axis of the maxillary central incisor and the SN reference plane and between the long axis of the maxillary central incisor and the FH reference plane, and an aesthetic relationship between lips and teeth.

Preferably, the step 4 includes:
step 401: determining a range of a maxillary functional region and a sagittal position of the maxillary canine based on a centric combination relationship between the target functional occlusal plane and the mandible of the treatment target position;
step 402: determining a horizontal width position of the maxillary canine based on frontal smile lip and tooth aesthetics;
step 403: determining a cusp inclination and an axis inclination of each of the canine and the first bicuspid based on an anatomical structure of a temporomandibular joint and an average value of the lateral condylar guidance;
step 404: determining a vertical position of the maxillary canine based on a compensating curve and the sagittal position of the maxillary canine;
step 405: obtaining the treatment target positions from the canine to the bicuspid in the middle section of the maxillary arch based on the sagittal position, the horizontal width position, the cusp inclination and axis inclination of each of the canine and first bicuspid, and the vertical position of the maxillary canine.

Preferably, the step 8 includes:
for a child with ideal mandibular structure and position, the centric relation of the child is obtained through clinical treatment; for a child who needs remote treatment or suffers from more severe functional deviation, asymmetric muscle tension or is too young to cooperate in obtaining the centric relation, if the anatomical form of the temporomandibular joint is normal but the structural position of the joint is abnormal, the mandibular position where the joint structure is symmetrical and within a reasonable range is obtained based on adjustment of the CBCT image, and the mandibular arch is adjusted based on the mandibular position to match the maxillary arch in the ideal target position.

Preferably, the step 8 includes:
for a child with mandibular retrusion and normal or low facial height, based on the CBCT image and on the premise of ensuring the symmetry of the left and right joint structures, the mandible is virtually moved forward and downward as a whole by moving the condylar process forward and downward along the condylar path to the lowest part of a posterior slope of the joint, where a total sagittal and vertical movement is controlled to range from 8 mm to 10 mm, a hard functional appliance is designed, manufactured and used in this mandibular position after virtual occlusal reconstruction to stabilize the adjusted mandibular position, and the invisible appliance is simultaneously used to move the teeth, so that the mandibular dentition at this mandibular position is matched with the dentition in the ideal target position of the maxilla.

Preferably, the step 8 includes:
for a child with a short mandibular ramus, a large anterior facial height and a small posterior facial height, and backward rotation of mandible and a high angle, the mandible can be virtually moved vertically downward, where a target movement amount is a distance from a mandibular foramen to an extension line of the target functional occlusal plane, and the vertical movement amount at a time is controlled to range from 4 mm to 5 mm, and in the molar segment space provided by occlusal reconstruction, with the ideal maxillary target dentition and functional occlusal plane as reference, the invisible appliance is used to extend the maxillary molars as quickly as possible to establish an ideal maxillary functional arch, and the anterior teeth are lowered, to change the inclination of the functional occlusal plane and the compensating curve, change the mandibular SPEE curve to match the compensating curve, to promote the counterclockwise growth and development of the mandible.

Preferably, the step 8 includes:
for children with functional crossbite, where those with underdeveloped maxillary structure should be excluded, if the maxillary structure of the child at a young age with underdeveloped maxillary structure is basically well developed after treatment or the crossbite is only a functional condition, the mandible can be virtually moved to release the locking of the anterior teeth in the vertical direction and id moved backward in the sagittal direction, where a virtual movement amount is based on an amount of posterior joint space measured by the CBCT, the total amount of sagittal and vertical movements is controlled to range from 5 mm to 6 mm, the invisible appliance or elastic one-piece jaw pad appliance is used to move the teeth in the maxillary and mandibular relationship after occlusal reconstruction to form a good occlusal relationship.

In a second aspect, an early treatment appliance for guiding mandibular growth and reconstruction with a maxillary occlusal structure is provided, which is manufactured by the method for manufacturing an early treatment appliance for guiding mandibular growth and reconstruction with the maxillary occlusal structure according to the above solution.

Compared with the conventional technology, the present application at least has the following beneficial effects.

The present application provides a method for manufacturing an early treatment appliance for guiding mandibular growth and reconstruction with a maxillary occlusal structure, where a maxillary arch is arranged by determining a target functional occlusal plane, a treatment target position of a maxillary first molar, a treatment target position of a maxillary central incisor, treatment target positions from canine to bicuspid in a middle section of the maxillary arch, and a dental arch form, and a mandibular arch is determined by obtaining a centric relation jaw position based on child's condition and the maxillary arch, the mandibular position may be virtually adjusted based on the child's condition, and then a functional appliance combined with an invisible appliance or a one-piece jaw pad is used to guide the mandible to match the maxilla at an occlusal reconstruction position to form the target mandibular arch. The early treatment appliance manufactured by the method for manufacturing the early treatment appliance provided in the present application can timely block the abnormalities of the maxillary structure and occlusal relationship based on the condition of the child, control the development of oral and facial deformities, and restore the maxillary arch structure to a normal range that conforms to the individual's age and growth pattern while controlling pathogenesis, and at the same time eliminate severe abnormal occlusal interference and guide mandibular growth by utilizing growth potential and the strength of chewing muscle movement, so that the early orthodontic treatment of the mixed dentition and early-stage permanent dentition is effective, the treatment period is short, and the efficiency is high, which helps the children to recover as soon as possible, and is suitable for the orthodontic treatment of all children with mixed dentition and early-stage permanent dentition before the mandibular development is completed.

### BRIEF DESCRIPTION OF THE DRAWINGS

In order to illustrate the conventional technology and the present application more intuitively, several exemplary drawings are provided. It should be understood that the specific shapes and structures shown in the drawings should not be generally considered as the limiting conditions for realizing the preset application. For example, based on the technical concept and exemplary drawings disclosed in the present application, those skilled in the art have the ability to easily make routine adjustments or further optimization on the division of addition/subtraction/attribution, specific shape, positional relationship, connection method, size proportion relationship, of some units (components).

FIG. 1 is a flow chart showing a method for manufacturing an early treatment appliance for guiding mandibular growth and reconstruction with a maxillary occlusal structure according to a first embodiment of the present application

### DETAILED DESCRIPTION OF THE EMBODIMENTS

The present application is further described in detail through specific embodiments in conjunction with the accompanying drawings.

In the present application, the word “multiple” indicates two or more unless otherwise specified. The terms “first”, “second” and “third” used in the present application are intended to distinguish the objects referred to and do not have special significance of technical connotation (for example, should not be understood as emphasizing on importance or order). Expressions such as “include”, “contain” and “have” also mean “not limited to” (some units, parts, materials, steps, etc.).

The terms used in the present application, such as “upper”, “lower”, “left”, “right” and “middle”, are generally for the convenience of intuitive understanding with reference to the attached drawings, and are not absolute limitations on the positional relationship in actual product. Without departing from the technical concept disclosed in the present application, changes to these relative positional relations should also be considered within the scope of the expression in the present application.

For children with maxillofacial deformities and oral malocclusion, if the abnormalities in the maxillary structure and occlusal relationship can be blocked in time, the development of oral and maxillofacial deformities can be controlled, where whether the morphological structure of the maxillary arch is normal is a key factor. If the maxillary arch structure can be restored to the normal range that is consistent with the individual's age and growth pattern while controlling the pathogenesis, and at the same time, severe abnormal occlusal interference is eliminated and the growth potential and the power of chewing muscle movement are used to guide the mandibular growth, the orthodontic treatment for the mixed dentition and early-stage permanent dentition can be effective, the treatment period is short, and the efficiency is higher. A short treatment period enables the child to recover to have healthy chewing, swallowing, breathing and pronunciation as soon as possible, thereby ensuring the child's overall health. Based on this, the present application provides a method for manufacturing an early treatment appliance for guiding mandibular growth and reconstruction with a maxillary occlusal structure.

### First Embodiment

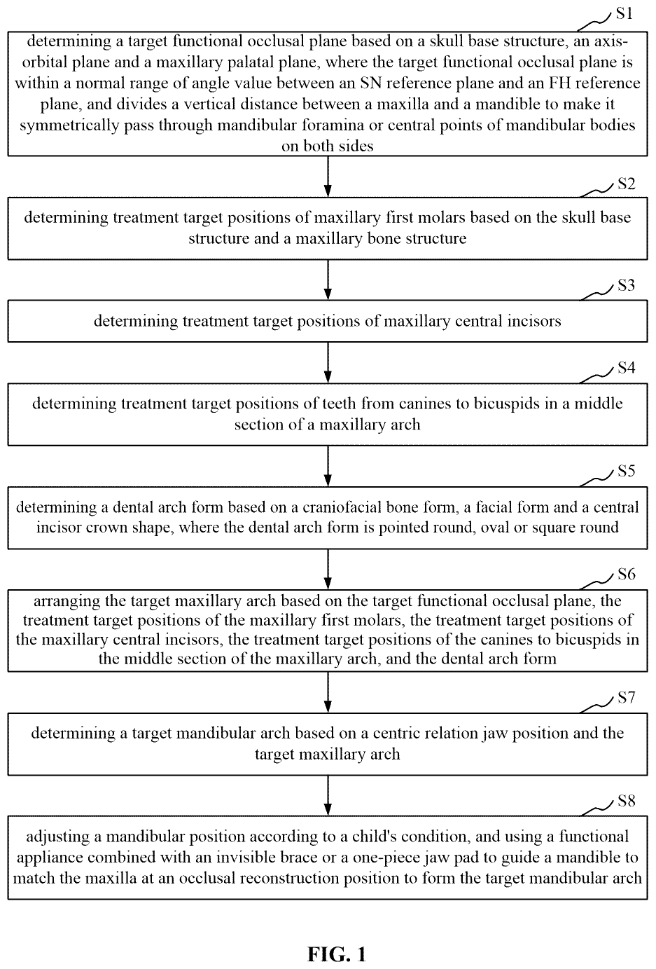

Referring to FIG. 1, a method for manufacturing an early treatment appliance for guiding mandibular growth and reconstruction with a maxillary occlusal structure is provided according to the first embodiment, including the following steps.
Step 1: determining a target functional occlusal plane based on a skull base structure, an axis-orbital plane and a maxillary palatal plane.
Step 101: determining ideal positions of a maxillary point A and a mandibular point B based on the skull base structure, the axis-orbital plane and the maxillary palatal plane, to obtain a target AB plane.
Specifically, in orthodontics, the point A and point B refer to two specific anatomical structures in the jaw, which are used to evaluate and measure the position and relationship of maxillofacial bones. The point A (Subspinale) is a most concave point on the bone between an anterior nasal spine and a superior prosthion, and the point B (Supramental) is a most concave point on the bone between between an infradentale and a pogonion.
Step 102: setting an inclination of the functional occlusal plane to make it be perpendicular to the target AB plane, and adjusting within a normal range of angle value of an SN reference plane and an FH reference plane based on the skull base structure and growth pattern. The target functional occlusal plane is within a normal range of angle value of the SN reference plane and the FH reference plane, and divides a vertical distance between the maxilla and the mandible to make it symmetrically pass through central points of mandibular foramina or mandibular bodies on both sides.
Specifically, the SN (Sella-Nasion) reference plane is a plane formed by connecting a mid-point of a sella turcica image and an anterior point of a frontonasal suture, and the FH (Frankfurt-Horizontal) reference plane is a plane formed by connecting an uppermost point of an external auditory meatus and a lowest point of an infraorbital margin.
Step 103: dividing the vertical distance between the maxilla and the mandible to make it symmetrically pass through the central points of the mandibular bodies or the mandibular foramina on both sides, thereby obtaining the final target functional occlusal plane.
Step 2: determining a treatment target position of a maxillary first molar based on the skull base structure and a maxillary bone structure.
Step 201: determining a width of a buccal wall of a mesiobuccal cusp of the maxillary first molar based on a width of the skull base structure and a width of the maxillary base.
Step 202: determining a sagittal position, a vertical position and a mesiodistal inclination of the maxillary first molar based on that a tooth long axis of the maxillary first molar passing through the mesiobuccal cusp is perpendicular to the target functional occlusal plane and an extension line of the tooth long axis of the maxillary first molar passing through the mesiobuccal cusp passes through a KR point.
Specifically, when determining the sagittal position, the vertical position and the mesiodistal inclination of the maxillary first molar, distances from condylar centers on both sides to the mesiobuccal cusps of the maxillary first molars are set to be the same.
It should be noted that the “KR (Key ridge) point” in this step refers to a lowest point of the KR (zygomatic process line) zygomatic bone contour, which is the point where the bite force is concentrated on the zygomatic process.
Step 203: determining a buccolingual torque of the maxillary first molar based on an ideal curve of Wilson.
Step 204: determining a rotation degree of the maxillary first molar based on an arch width, an arch curvature and a position of a contralateral canine.
Step 205: obtaining a treatment target position of the maxillary first molar based on the width, the sagittal position, the vertical position, the mesiodistal inclination, the buccolingual torque and the rotation degree.
Step 3: determining a treatment target position of a maxillary central incisor.
Step 301: determining a radius range of a midpoint of a palatine fossa of the maxillary central incisor based on an equilateral triangle formed by a distance between the condylar centers on both sides of a CBCT image;
Specifically, a facial mandibular angle (FMA) is adjusted to the normal range based on the growth pattern of the child, and the ideal mandibular plane is obtained by combining the maxillary palatal plane and the AB plane. The parallel plane of the ideal mandibular plane passes through the left and right condylar centers and forms an equilateral triangle or an isosceles triangle with the symmetrical left and right condylar centers. A vertex of this triangle is the BP point, that is, the midpoint of the palatine fossa of the maxillary central incisor in the ideal target position of the maxillary arch. For children with horizontal growth pattern and vertical growth pattern, the triangle can be slightly adjusted to be an isosceles triangle to make the BP point more consistent with children's growth and development.
Step 302: determining a sagittal position and a vertical position of the maxillary central incisor of an ideal treatment target position of the maxillary arch based on the radius range, the target functional occlusal plane, a compensating curve and a maxillary central incisor closing axis.
Step 303: obtaining child's current condyle path data based on a CBCT glenoid fossa image.
Step 304: determining child's current incisal guidance and condylar guidance based on the child's age and the condyle path data.
Step 305: determining a treatment target position incisal path of the maxillary central incisor based on the incisal guidance, the condylar guidance, and the growth pattern.
Step 306: determining an inclination of the maxillary central incisor based on the midpoint of the palatine fossa of the maxillary central incisor, the treatment target position incisal path of the maxillary central incisor, and the palatine anatomical form of the maxillary central incisor.
Step 307: obtaining the treatment target position of the maxillary central incisor by fine-tuning the sagittal position and the vertical position of the maxillary central incisor and the inclination of the maxillary central incisor with reference to a correlation between a height of contour of the maxillary central incisor and a facial profile, angles between a long axis of the maxillary central incisor and the SN reference plane and between the long axis of the maxillary central incisor and the FH reference plane, and an aesthetic relationship between the lips and teeth.
Step 4: determining treatment target positions from the canine to the bicuspid in the middle section of the maxillary arch.
Step 401: determining a range of a maxillary functional region and a sagittal position of the maxillary canine based on a centric combination relationship between the target functional occlusal plane and the mandible of the treatment target position.
Step 402: determining a horizontal width position of the maxillary canine based on a frontal smile lip and tooth aesthetics.
Step 403: determining a cusp inclination and an axis inclination of each of the canine and the first bicuspid based on an anatomical structure of a temporomandibular joint and an average value of the lateral condylar guidance.
Step 404: determining a vertical position of the maxillary canine based on a compensating curve and the sagittal position of the maxillary canine.
Step 405: obtaining the treatment target positions from the canine to the bicuspid in the middle section of the maxillary arch based on the sagittal position, the horizontal width position, the cusp inclination and axis inclination of each of the canine and first bicuspid, and the vertical position of the maxillary canine.
Step 5: determining the dental arch form based on a craniofacial bone form, a facial form and a central incisor crown shape.
Specifically, the dental arch form is pointed round, oval or square round.
Step 6: arranging the target maxillary arch based on the target functional occlusal plane, the treatment target position of the maxillary first molar, the treatment target position of the maxillary central incisor, the treatment target positions of the canine to bicuspid in the middle section of the maxillary arch, and the dental arch form.
Specifically, when arranging the maxillary arch, it is necessary to deal with the torsion and contact relationship of the teeth, to form active and passive functional arches that are bilaterally symmetrical with the midpalatal suture as the central axis and vertically symmetrical in height with the orbito-auricular plane or axis-orbital plane as the distance, ultimately obtaining the maxillary compensation curve and curve of Wilson that meet the occlusal function.
Therefore, the target maxillary arch formed in this step is horizontally symmetrical with the midpalatal suture as the central axis, and the target maxillary arch formed in this step is vertically symmetrical in height with the line connecting the left and right condylar centers as a reference. The left and right molar central fossae, molar mesial marginal ridges, bicuspid central fossae and canine incisor palatine fossae of the target maxillary arch form a continuous symmetrical arc.
Step 7: determining a target mandibular arch based on the centric relation jaw position and the target maxillary arch.
Specifically, in order to obtain a normal and healthy occlusal relationship, the long axis of the mandibular first molar passing through the buccal groove must be perpendicular to the functional occlusal plane and coincide with the long axis of the maxillary first molar passing through the maxillary mesiobuccal cusp. In static occlusion, three-point contact is formed between the mandibular molars and the maxillary molars (where the maxillary contact points are the palatal cusp, the mesial marginal ridge, and central fossa, and the mandibular contact points are the mesial and distal buccal cusps and central fossa), two-point contact is formed between the mandibular bicuspid and the maxillary bicuspid (where the maxillary contact points are the palatal cusp and the mesial marginal ridge, and the mandibular contact points are the distal marginal ridge and the buccal cusp), and the mandibular anterior teeth form light contact at the marginal ridge next to the palatine fossa of the maxillary central incisor and lateral incisor, forming a Class I relationship between molars and canines and a normal overbite and overjet of the anterior teeth. The mandibular SPEE curve is consistent with the compensating curve, and the maxillary and mandibular curve of Wilsons are consistent. During protrusive movement, the anterior teeth have light contact along the incisal path while the posterior teeth have no contact. During lateral movement, the working side canines have contact while the other teeth and the non-working side teeth have no contact (the canines are replaced by the first bicuspids or deciduous canines before they erupt). The occlusal separation angle during the movement is between 8 degrees to 10 degrees, and the molars can be separated immediately at the initial stage of mouth opening movement without occlusal interference.
Step 8: adjusting a mandibular position based on the child's condition, and using a functional appliance combined with an invisible appliance or a one-piece jaw pad to guide the mandible to match the maxilla at the occlusal reconstruction position to form the target mandibular arch.
Specifically, for a child with ideal mandibular structure and position, the centric relationship of the child is obtained through clinical treatment. For a child who needs remote treatment or suffers from more severe functional deviation, asymmetric muscle tension or is too young to cooperate in obtaining the centric relation, it is difficult to make correct occlusal reconstruction clinically. If the anatomical form of the temporomandibular joint is normal but the structural position of the joint is abnormal, the mandibular position where the joint structure is symmetrical and within a reasonable range is obtained based on adjustment of the CBCT image, and the mandibular arch is adjusted based on the mandibular position to match the maxillary arch in the ideal target position.

For a child with mandibular retrusion and normal or low facial height, based on the CBCT image and on the premise of ensuring the symmetry of the left and right joint structures, the mandible is virtually moved forward and downward as a whole by moving the condylar process forward and downward along the condylar canal to the lowest part of a posterior slope of the joint, where a total sagittal and vertical movement is controlled to be 8 to 10 mm. A hard functional appliance is designed, manufactured and used in this mandibular position after virtual occlusal reconstruction to stabilize the adjusted mandibular position, and the invisible appliance is simultaneously used to move the teeth, so that the mandibular dentition at this mandibular position is matched with the dentition in the ideal target position of the maxilla.

For a child with a short mandibular ramus, a large anterior facial height and a small posterior facial height, and backward rotation of mandible and a high angle, the mandible can be virtually moved vertically downward. The target movement amount is the distance from the mandibular foramen to the extension line of the target functional occlusal plane. The vertical movement amount at a time is controlled to range from 4 to 5 mm. In the molar segment space provided by occlusal reconstruction, with the ideal maxillary target dentition and functional occlusal plane as reference, the invisible appliance is used to extend the maxillary molars as quickly as possible, to establish an ideal maxillary functional arch, and the anterior teeth are lowered, to change the inclination of the functional occlusal plane and the compensating curve, change the mandibular SPEE curve to match the compensating curve, to promote the counterclockwise growth and development of the mandible.

For children with functional crossbite, those with underdeveloped maxillary structure should to be excluded. If the maxillary structure of a child at a young age with underdeveloped maxillary structure is basically well developed after treatment or the crossbite is only a functional condition, the mandible can be virtually moved to release the locking of the anterior teeth in the vertical direction and move backward in the sagittal direction. The virtual movement amount is based on the amount of posterior joint space measured by CBCT. The total amount of sagittal and vertical movements is controlled to range from 5 to 6 mm. An invisible appliance or elastic one-piece jaw pad appliance are used to move the teeth in the maxillary and mandibular relationship after occlusal reconstruction to form a good occlusal relationship.

In the method for manufacturing an early treatment appliance for guiding mandibular growth and reconstruction with a maxillary occlusal structure provided in this embodiment, a jaw pad may be provided to guide mandibular occlusal reconstruction based on the child's condition.

Specifically, at a stage where the dentition does not move but the mandibular repositioning is required in a large range, virtual occlusal reconstruction is performed as needed, and the inter-maxillary space of the occlusal reconstruction is taken to make a hard anatomical jaw pad, which is fixed to the maxillary dentition and worn all day including eating. During the treatment, the occlusal contact surface of the jaw pad is adjusted to guide the mandibular growth.

When the occlusal interference needs to be relieved for functional restoration of the mandible, a flat jaw pad or a semi-anatomical jaw pad can be combined with the invisible or functional appliance to help bite opening and restore the mandible to a functional position.

In a stage where the dentition needs to move a little or do not need to move and the mandibular repositioning is required in a small range, or in a stage of inducing lateral dentition replacement of deciduous and permanent teeth or in a maintenance stage after orthodontic treatment, virtual occlusion reconstruction can be performed as needed, where the vertical open space of the upper and lower dentition of the ideal target occlusion is taken, and a virtual flat jaw pad is arranged in the middle area and load-bearing area of the dentition. The flat jaw pad is used to process the cusps of the middle and posterior sections of the molars to form the necessary curvature and thickness, and an elastic resin one-piece orthodontic appliance is made to fine-tune the position of the dentition and mandible through the muscle strength of biting and swallowing. The design of the lip and tongue stop of the one-piece orthodontic appliance cooperates with the clinical wearing method, to improve the coordination of the perioral muscles and the muscles for opening and closing the mouth.

The early treatment appliance manufactured by the method for manufacturing an early treatment appliance for guiding mandibular growth and reconstruction with a maxillary occlusal structure provided in the present embodiment can timely block the abnormalities of the maxillary structure and occlusal relationship based on the condition of the child, control the development of oral and facial deformities, and restore the maxillary arch structure to a normal range that conforms to the individual's age and growth pattern while controlling the pathogenesis, and at the same time eliminate severe abnormal occlusal interference and guide mandibular growth by utilizing the growth potential and the power of chewing muscle movement, so that the orthodontic treatment of the mixed dentition and early-stage permanent dentition is effective, the treatment period is short, and the efficiency is high, which helps the children to recover as soon as possible, and is suitable for the orthodontic treatment of all children with mixed dentition and early-stage permanent dentition before the mandibular development is completed.

### Second Embodiment

This embodiment provides an early treatment appliance for guiding mandibular growth and reconstruction with a maxillary occlusal structure, which is manufactured according to a method for manufacturing an early treatment appliance for guiding mandibular growth and reconstruction with a maxillary occlusal structure provided by the first embodiment.

Technical features of the foregoing embodiments can be combined freely (as long as there are no contradictions in the combinations of these technical features). For conciseness of description, not all possible combinations of the technical features of the foregoing embodiments are described. However, the embodiments that are not explicitly written shall fall within the scope of this specification.

The present application has been described in detail through general description and specific embodiments. It should be understood that based on the technical concept of the present application, some conventional adjustments or further innovations can be made to these specific embodiments. However, as long as not deviating from the technical concept of the present application, the technical solutions obtained by these conventional adjustments or further innovations also fall within the scope of protection of the claims of the present application.

What is claimed is:

1. A method for manufacturing an early treatment appliance for guiding mandibular growth and reconstruction with a maxillary occlusal structure, comprising:
step 1: determining a target functional occlusal plane based on a skull base structure, an axis-orbital plane and a maxillary palatal plane, wherein the target functional occlusal plane is within a normal range of angle value between an Sella-Nasion (SN) reference plane and an Frankfort horizontal (FH) reference plane, and divides a vertical distance between a maxilla and a mandible to make it symmetrically pass through mandibular foramina or central points of mandibular bodies on both sides;
step 2: determining a treatment target position of a maxillary first molar based on the skull base structure and a maxillary bone structure;
step 3: determining a treatment target position of a maxillary central incisor;
step 4: determining treatment target positions of canine to bicuspid in a middle section of a maxillary arch;
step 5: determining a dental arch form based on a craniofacial bone form, a facial form and a central incisor crown shape, wherein the dental arch form is pointed round, oval or square round;
step 6: arranging a target maxillary arch based on the target functional occlusal plane, the treatment target position of the maxillary first molar, the treatment target position of the maxillary central incisor, the treatment target positions of the canine to the bicuspid in the middle section of the maxillary arch, and the dental arch form;
step 7: determining a target mandibular arch based on a centric relation jaw position and the target maxillary arch; and
step 8: adjusting a mandibular position according to a child's condition, and using a functional appliance combined with an invisible appliance or a one-piece jaw pad to guide the mandible to match the maxilla at an occlusal reconstruction position to form the target mandibular arch.

2. The method according to claim 1, wherein the step 1 comprises:
step 101: determining ideal positions of a maxillary point A and a mandibular point B based on the skull base structure, the axis-orbital plane and the maxillary palatal plane, to obtain a target anterior cranial base (AB) plane;
step 102: setting an inclination of the functional occlusal plane to make it be perpendicular to the target AB plane, and performing adjustment within the normal range of the angle value between the SN reference plane and the FH reference plane based on the skull base structure and growth pattern; and
step 103: dividing the vertical distance between the maxilla and the mandible to make it symmetrically pass through the central points of the mandibular bodies or the mandibular foramina on both sides, to obtain the final target functional occlusal plane.

3. The method according to claim 1, wherein the step 2 comprises:
step 201: determining a width of a buccal wall of a mesiobuccal cusp of the maxillary first molar based on a width of the skull base structure and a width of the maxillary base;
step 202: determining a sagittal position, a vertical position and a mesiodistal inclination of the maxillary first molar based on that a tooth long axis of the maxillary first molar passing through the mesiobuccal cusp is perpendicular to the target functional occlusal plane and an extension line of the tooth long axis of the maxillary first molar passing through the mesiobuccal cusp passes through a key ridge (KR) point, wherein when determining the sagittal position, the vertical position and the mesiodistal inclination of the maxillary first molar, distances from condylar centers on both sides to the mesiobuccal cusps of the maxillary first molars are set to be the same;
step 203: determining a buccolingual torque of the maxillary first molar based on an ideal curve of Wilson;
step 204: determining a rotation degree of the maxillary first molar based on an arch width, an arch curvature and a position of a contralateral canine; and
step 205: obtaining the treatment target position of the maxillary first molar based on the width, the sagittal position, the vertical position, the mesiodistal inclination, the buccolingual torque and the rotation degree.

4. The method according to claim 1, wherein the step 3 comprises:
step 301: determining a radius range of a midpoint of a palatine fossa of the maxillary central incisor based on an equilateral triangle formed by a distance between the condylar centers on both sides of a cone beam computed tomography (CBCT) image;
step 302: determining a sagittal position and a vertical position of the maxillary central incisor of an ideal treatment target position of the maxillary arch based on the radius range, the target functional occlusal plane, a compensating curve and a maxillary central incisor closing axis;
step 303: obtaining child's current condyle path data based on a CBCT glenoid fossa image;
step 304: determining child's current incisal guidance and condylar guidance based on the child's age and the condyle path data;
step 305: determining a treatment target position incisal path of the maxillary central incisor based on the incisal guidance, the condylar guidance, and the growth pattern;
step 306: determining an inclination of the maxillary central incisor based on the midpoint of the palatine fossa of the maxillary central incisor, the treatment target position incisal path of the maxillary central incisor, and palatine anatomical form of the maxillary central incisor; and
step 307: obtaining the treatment target position of the maxillary central incisor by fine-tuning the sagittal position and the vertical position of the maxillary central incisor and the inclination of the maxillary central incisor with reference to a correlation between a height of contour of the maxillary central incisor and a facial profile, angles between a long axis of the maxillary central incisor and the SN reference plane and between the long axis of the maxillary central incisor and the FH reference plane, and an aesthetic relationship between lips and teeth.

5. The method according to claim 1, wherein the step 4 comprises:
step 401: determining a range of a maxillary functional region and a sagittal position of the maxillary canine based on a centric combination relationship between the target functional occlusal plane and the mandible of the treatment target position;
step 402: determining a horizontal width position of the maxillary canine based on frontal smile lip and tooth aesthetics;
step 403: determining a cusp inclination and an axis inclination of each of the canine and the first bicuspid based on an anatomical structure of a temporomandibular joint and an average value of the lateral condylar guidance;
step 404: determining a vertical position of the maxillary canine based on a compensating curve and the sagittal position of the maxillary canine; and
step 405: obtaining the treatment target positions from the canine to the bicuspid in the middle section of the maxillary arch based on the sagittal position, the horizontal width position, the cusp inclination and axis inclination of each of the canine and first bicuspid, and the vertical position of the maxillary canine.

6. The method according to claim 1, wherein the step 8 comprises:
obtaining the centric relation of ta child is obtained through clinical treatment for the child with ideal mandibular structure and position; alternatively,
obtaining a mandibular position where a joint structure is symmetrical and within a reasonable range based on adjustment of a cone beam computed tomography (CBCT) image when an anatomical form of a temporomandibular joint is normal but a structural position of the joint is abnormal for another child who needs remote treatment or suffers from more severe functional deviation, asymmetric muscle tension or is too young to cooperate in obtaining the centric relation; and
adjusting the mandibular arch based on the mandibular position to match the maxillary arch in the ideal target position.

7. The method according to claim 1, wherein the step 8 comprises:
for a child with mandibular retrusion and normal or low facial height, based on a cone beam computed tomography (CBCT) image and on the premise of ensuring symmetry between left and right joint structures, the mandible is virtually moved forward and downward as a whole by moving a condylar process forward and downward along the condylar path to the lowest part of a posterior slope of a joint, wherein a total sagittal and vertical movement is controlled to range from 8 mm to 10 mm, a hard functional appliance is designed, manufactured and used in the mandibular position after virtual occlusal reconstruction, to stabilize the adjusted mandibular position, and the invisible appliance is simultaneously used to move the teeth, to make a mandibular dentition be matched with a maxilla dentition in the ideal target position.

8. The method according to claim 1, wherein the step 8 comprises:
for a child with a short mandibular ramus, a large anterior facial height and a small posterior facial height, and backward rotation of mandible and a high angle, the mandible is virtually moved vertically downward, wherein a target movement amount is a distance from a mandibular foramen to an extension line of the target functional occlusal plane, and a vertical movement amount at a time is controlled to range from 4 to 5 mm, and in a molar segment space provided by occlusal reconstruction, with an ideal maxillary target dentition and the functional occlusal plane as reference, the invisible appliance is used to extend the maxillary molars as quickly as possible, to establish an ideal maxillary functional arch, and the anterior teeth are lowered, to change an inclination of the functional occlusal plane and the compensating curve, change a mandibular SPEE curve to match the compensating curve, to promote counterclockwise growth and development of the mandible.

9. The method according to claim 1, wherein the step 8 comprises:
for children with functional crossbite, wherein those with underdeveloped maxillary structure should to be excluded, if the maxillary structure of a child at a young age with underdeveloped maxillary structure is basically well developed after treatment or the crossbite is only a functional condition, the mandible is virtually moved to release the locking of anterior teeth in the vertical direction and is moved backward in the sagittal direction, wherein a virtual movement amount is based on an amount of posterior joint space measured by CBCT, a total amount of sagittal and vertical movements is controlled to range from 5 mm to 6 mm, the invisible appliance or elastic one-piece jaw pad appliance is used to move teeth in an maxillary and mandibular relationship after occlusal reconstruction, to form a good occlusal relationship.

10. An early treatment appliance for guiding mandibular growth and reconstruction with a maxillary occlusal structure, manufactured by the method according to claim 1.
